(19) United States
(12) Patent Application Publication
(10) Pub. No.: US 2010/0247935 A1
(43) Pub. Date: Sep. 30, 2010

(54) NON-PVC FILMS HAVING BARRIER LAYER

(75) Inventors: Moh-Ching Oliver Chang, Lake In The Hills, IL (US); Yuanpang S. Ding, Libertyville, IL (US); Michael Tung-Kiung Ling, Vernon Hills, IL (US); John W. Putnam, Lindenhurst, IL (US); Robert R. Roberts, Burlington, WI (US); Fabio Casartelli, Delebio, Sondrio (IT); Jean-Claude Bonte, Lessines (BE); Patrick R. Balteau, Evelette (BE)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/409,644

(22) Filed: Mar. 24, 2009

(51) Int. Cl. B32B 27/08; B32B 27/34; A61B 19/00; B65D 30/02

(52) U.S. Cl. 428/476.1; 428/475.5; 604/410

(57) ABSTRACT

Films having peel seal layers and/or barrier layers are provided. In a general embodiment, the present disclosure provides a film comprising a barrier layer comprising a caprolactam-free nylon compound.

### BACKGROUND

The present disclosure relates generally to polymer films. More particularly, the present disclosure relates to non-PVC polymer films comprising novel peel seal and/or barrier layers.

Multilayer coextruded films are widely used throughout a variety of industries, for example, including use in containers for food or medical solution packaging. One of the desired properties of a multilayer extruded in film is its toughness or ability to resist damage in use or transport. Another desired property is the ability to make both a peel seal at the desired strength to suit the application as well as a permanent seal to permanently enclose a container. An additional desired property is to provide a barrier to gases such as oxygen, carbon dioxide or water vapor in order to maintain the stability of contained solutions.

Traditional flexible polyvinyl chloride materials have also typically been used to fabricate medical grade containers. Polyvinyl chloride (“PVC”) is a cost effective material for constructing such devices. However, PVC may generate objectionable amounts of hydrogen chloride (or hydrochloric acid when contacted with water) upon incineration. PVC sometimes contains plasticizers that may leach into drugs or biological fluids or tissues that come in contact with PVC formulations.

### SUMMARY

The present disclosure generally relates to films having peel seal layers and/or barrier layers. In a general embodiment, the present disclosure provides a film comprising a peel seal layer comprising a blend of a polypropylene (PP) random copolymer having a melting temperature greater than 140° C., a styrene-ethylene-butylene-styrene block copolymer (SEBS) and a linear low-density polyethylene (LLDPE) having a melting temperature greater than 115° C.

In an embodiment, the blend comprises about 60% to about 80% by weight of a polypropylene random copolymer having a melting temperature greater than 140° C., about 15% to about 30% by weight of a styrene-ethylene-butylene-styrene block copolymer, and about 2.5% to about 20% by weight of an LLDPE having a melting temperature greater than 115° C.

In another embodiment, the blend comprises about 70% by weight of a polypropylene random copolymer having a melting temperature greater than 145° C., about 22.5% by weight of a styrene-ethylene-butylene-styrene block copolymer, and about 7.5% by weight of an LLDPE having a melting temperature greater than 120° C. The LLDPE can be ethylene-octene-1 copolymer, ethylene-hexene-1 copolymer, or a combination thereof.

In an embodiment, the film comprises a skin layer and a barrier layer. For example, the skin layer and the peel seal layer can be attached to the barrier layer on opposing sides of the barrier layer. The skin layer can comprise a random copolymer polypropylene, homo-polymer polypropylene, polypropylene based TPO, nylon, styrene-ethylene-butylene-styrene block copolymer, copolyester ether, or a combination thereof. The barrier layer can comprise polyamide (nylon), for example polyamide 6,6/6,10 copolymer, polyamide 6, amorphous polyamide, rubber modified Nylon, or a combination thereof.

In an embodiment, the film comprises at least one tie layer that attaches at least one of the skin layer and the peel seal layer to the barrier layer. The tie layer can comprise maleated LLDPE, maleated polypropylene homo-polymer, maleated polypropylene copolymer, maleated TPO, or a combination thereof.

In another embodiment, the present disclosure provides a film comprising a peel seal layer comprising a blend of a polypropylene random copolymer having a melting temperature greater than 140° C. and an ethylene-propylene rubber modified polypropylene elastomer. The blend can comprise about 20% to about 40% by weight of the polypropylene random copolymer and about 60% to about 80% by weight of the ethylene-propylene rubber modified polypropylene elastomer.

In an embodiment, the film can comprise a skin layer, seal layer, and a barrier layer. The skin layer and the peel seal layer can be attached to the barrier layer on opposing sides of the barrier layer. The skin layer can comprise polypropylene homopolymer, polypropylene random copolymer, polypropylene based TPO, polyamide (nylon), styrene-ethylene-butylene-styrene block copolymer, copolyester ether copolymer, or a combination thereof. The barrier layer can comprise one or more polyamides (nylon), such as polyamide 6, polyamide 6,6/6,10 copolymer, amorphous polyamide, rubber modified, or a combination thereof. The film can further comprise at least one tie layer that attaches at least one of the skin layer and the peel seal layer to the barrier layer.

In an alternative embodiment, the present disclosure provides a film comprising a barrier layer comprising a caprolactam-free nylon compound. The caprolactam-free nylon compound can comprise a blend of about 75% to about 95% by weight of a polyamide 6,6/6,10 copolymer and about 5% to about 25% by weight of amorphous polyamide. In another embodiment, the caprolactam-free nylon compound comprises a blend of about 87.5% by weight of a polyamide 6,6/6,10 copolymer and about 12.5% by weight of amorphous polyamide.

In an embodiment, the film having the caprolactam-free nylon barrier layer can comprise a skin layer and a peel seal layer. The skin layer and the peel seal layer can be attached to the barrier layer on opposing sides of the barrier layer. The skin layer can comprise polypropylene homopolymer, polypropylene random copolymer, polypropylene based TPO, polyamide (nylon), styrene-ethylene-butylene-styrene block copolymer, copolyester ether block copolymer, or a combination thereof. The peel seal layer can comprise a blend of a polypropylene random copolymer having a melting temperature greater than 140° C., a styrene-ethylene-butylene-styrene block copolymer and an LLDPE having a melting temperature greater than 120° C. The film can further comprise at least one tie layer that attaches at least one of the skin layer and the peel seal layer to the barrier layer.

In an embodiment, the film may include a core layer positioned between the skin layer and the peel seal layer, for example between the skin layer and the barrier layer or between the peel seal layer and the barrier layer. The core layer may contain propylene-ethylene copolymer, syndiotactic propylene-ethylene copolymer, polypropylene elastomer, polypropylene homopolymer, propylene based elastomer, ethylene based elastomer, styrene-ethylene-butylene-styrene block copolymer, ethylene-propylene rubber modified polypropylene, or a combination thereof.

In another embodiment, the film can be used to make any suitable container, for example, used to hold a substance such as a pharmaceutical or a medical compound or solution. The present disclosure provides a container comprising a first sidewall and a second sidewall sealed together along at least one peripheral edge to define a fluid chamber. At least one of the first and second sidewall of the container is a film comprising at least one of 1) a peel seal layer comprising a blend of a polypropylene random copolymer having a melting temperature greater than 140° C., a styrene-ethylene-butylene-styrene block copolymer and an LLDPE having a melting temperature greater than 115° C.; 2) a peel seal layer comprising a blend of a polypropylene random copolymer having a melting temperature greater than 140° C. and an ethylene-propylene rubber modified polypropylene elastomer; and 3) a barrier layer comprising a caprolactam-free nylon compound.

In an alternative embodiment, the present disclosure provides a multiple chamber container comprising a body defined by a film. The body can include two or more chambers separated by a peelable seal. The film can comprise at least one of 1) a peel seal layer comprising a blend of a polypropylene random copolymer having a melting temperature greater than 140° C., a styrene-ethylene-butylene-styrene block copolymer and an LLDPE having a melting temperature greater than 115° C.; 2) a peel seal layer comprising a blend of a polypropylene random copolymer having a melting temperature greater than 140° C. and an ethylene-propylene rubber modified polypropylene elastomer; and 3) a barrier layer comprising a caprolactam-free nylon compound.

An advantage of the present disclosure is to provide improved non-PVC films.

Another advantage of the present disclosure is to provide improved peel seal layers for polymer films.

Yet another advantage of the present disclosure is to provide improved barrier layers for polymer films.

Still another advantage of the present disclosure is to provide improved methods of making non-PVC films.

Another advantage of the present disclosure is to provide improved containers comprising non-PVC films.

Additional features and advantages are described herein, and will be apparent from the following Detailed Description and the figures.

### BRIEF DESCRIPTION OF THE FIGURES

FIG. 1 is a cross-sectional view of a monolayer film in an embodiment of the present disclosure.

FIG. 2 is a cross-sectional view of a five-layer film in an embodiment of the present disclosure.

FIG. 3 is a cross-sectional view of a six-layer film in an embodiment of the present disclosure.

FIG. 4 is a cross-sectional view of a container fabricated from a film in an embodiment of the present disclosure.

FIG. 5 is a cross-sectional view of a multiple chamber container fabricated from a film in an embodiment of the present disclosure.

FIG. 6 is graph showing peel seal performance for multiple layer films.

FIG. 7 is graph showing peel seal performance for multiple layer films.

FIG. 8 is a graph showing typical peel seal curves for different film formulations.

FIGS. 9(a)-(c) are cross-sectional views of multiple layer films in an embodiment of the present disclosure.

### DETAILED DESCRIPTION

The present disclosure generally relates to non-PVC films having peel seal layers and/or barriers layers. The disclosure provides monolayer films as well as multilayer films useful for packaging applications.

The films in embodiments of the present disclosure have improved toughness and peel seal capability while maintaining good gas barrier properties. This can be accomplished through compounding materials to provide appropriate seal layers that provide the appropriate peel seal range as well as toughness along with choosing skin layers that improve the toughness of the film. In an embodiment, the peel seal layer and barrier layer films can have properties such as toughness or ability to absorb impact energy, sterilizability at 121° C., low haze, barrier to gases, peel sealability using heat sealing machinery and affordability.

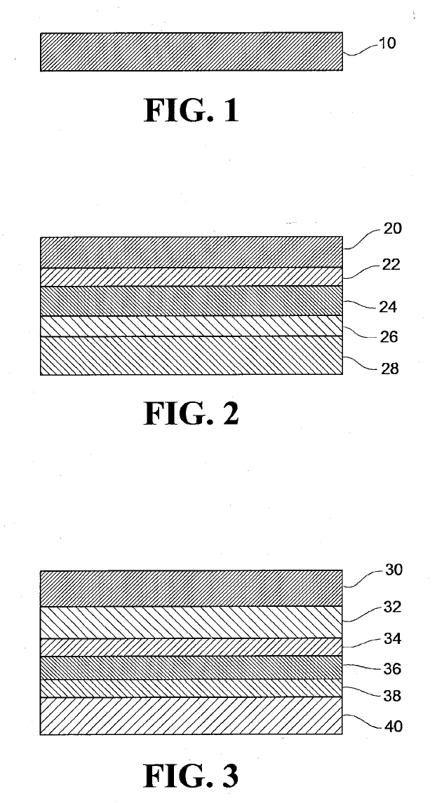

In a general embodiment illustrated in FIG. 1, the present disclosure provides a film 10 including a peel seal layer containing a blend of a random copolymer polypropylene (PP) having a melting temperature greater than 140° C., a styrene-ethylene-butylene-styrene block copolymer and an LLDPE having a melting temperature greater than 115° C. Suitable random copolymer polypropylenes include those sold by Flint Hills Resources under the HUNTSMAN tradename and Borealis under the BOREALIS and TOTAL tradenames. Suitable styrene-ethylene-butylene-styrene block copolymers include those sold by Kraton under the KRATON tradename. Suitable LLDPEs include those sold by Exxon under the EXXON tradename and Dow under the DOWLEX tradename.

In an embodiment, the peel seal layer blend contains about 60% to about 80% by weight of a random copolymer polypropylene having a melting temperature greater than 145° C., about 15% to about 30% by weight of a styrene-ethylene-butylene-styrene block copolymer and about 2.5% to about 20% by weight of an LLDPE having a melting temperature greater than 120° C. In another embodiment, the blend contains about 70% by weight of a random copolymer polypropylene having a melting temperature greater than 145° C., about 22.5% by weight of a styrene-ethylene-butylene-styrene block copolymer and about 7.5% by weight of an LLDPE having a melting temperature greater than 120° C. The LLDPE can be ethylene-octene-1 copolymer, ethylene-hexene-1 copolymer, or a combination thereof.

In an embodiment illustrated in FIG. 2, the film is a five-layer film having a skin layer 20, a barrier layer 24 and a peel seal layer 28. For example, the skin layer 20 and the peel seal layer 28 can be directly or indirectly attached to the barrier layer 24 on opposing sides of the barrier layer 24. The skin layer 20 can contain a random copolymer polypropylene, homo-polymer polypropylene, nylon, styrene-ethylene-butylene-styrene block copolymer, copolyester ether, or a combination thereof. The barrier layer can contain one or more polyamides (nylon), for example polyamide 6, polyamide 6,6/6,10 copolymer, amorphous polyamide, or a combination thereof. Suitable polypropylene homopolymers include those sold by Flint Hills Resources under the HUNTSMAN trade name. Suitable nylons include those sold by EMS under the GRIVORY and GRILON trade names. Suitable ethylene-propylene rubber modified polypropylene elastomers include those sold by Mitsubishi under the ZELAS tradename.

In an embodiment shown in FIG. 2, the multilayer film includes one or more tie layers 22 and 26 that are used to attach the skin layer 20 and/or the peel seal layer 28 to the barrier layer 24. The tie layers 22 and 26 can contain any suitable adhesive material such as, for example, maleated LLDPE, maleated polypropylene homopolymer, maleated polypropylene copolymer, maleated polypropylene based TPO, or a combination thereof.

In another embodiment, the present disclosure provides a film including a peel seal layer containing a blend of a polypropylene random copolymer having a melting temperature greater than 145° C. and an ethylene-propylene rubber modified polypropylene elastomer. The blend may contain about 20% to about 40% by weight of a random copolymer polypropylene having a melting temperature greater than 140° C. and about 60% to about 80% by weight of an ethylene-propylene rubber modified polypropylene elastomer.

In an embodiment, the aforementioned film may further include a skin layer and a barrier layer. The skin layer and the peel seal layer can be attached to the barrier layer on opposing sides of the barrier layer. The skin layer may contain polypropylene homopolymer, polypropylene random copolymer, polypropylene based elastomer, polyamide (nylon), styrene-ethylene-butylene-styrene block copolymer, copolyester ether block copolymer, or a combination thereof. The barrier layer can include one or more polyamides (nylon), for example polyamide 6, polyamide 6,6/6,10 copolymer, amorphous polyamide, or a combination thereof. The film can further include at least one tie layer that attaches at least one of the skin layer and the peel seal layer to the barrier layer.

Films including a barrier layer in embodiments of the present disclosure may contain a caprolactam-free nylon barrier material with good gas barrier resistance and adequate toughness for use in multilayer films for medical solution container applications. Traditionally, nylon-6 (polyamide-6) or nylon-6 based blends have provided a good combination of gas barrier and impact resistance. However, nylon-6 cannot be used for plastic solution container applications in certain countries such as Japan, Korea, and China due to their pharmacopoeia requirements. The pharmacopoeia requirements in these countries include limitations on the UV/visible light wavelengths of compounds extracted into solution from the container film materials; these limitations severely limit the permissible amount of caprolactam in the container film. Because polyamide-6 is synthesized from caprolactam, its presence will cause the container film to fail the criteria when used at a thickness that provides useful barrier properties.

In an alternative embodiment, the present disclosure provides a film including a barrier layer containing a caprolactam-free nylon (i.e. polyamide or PA) compound. The caprolactam-free nylon compound can comprise a blend of about 75% to about 95% by weight of a polyamide 6,6/6,10 copolymer and about 5% to about 25% by weight of amorphous polyamide. In another embodiment, the caprolactam-free nylon compound comprises a blend of about 87.5% by weight of a polyamide 6,6/6,10 copolymer and about 12.5% by weight of amorphous polyamide. Suitable amorphous polyamides include, without limitation, polyamide 6I/6T and polyamide MXD6/MXDI copolymer.

In an embodiment illustrated in FIG. 3, the film includes a skin layer 30, caprolactam-free nylon barrier layer 36 and a peel seal layer 40. The skin layer 30 and the peel seal layer 40 can be attached to the barrier layer 36 on opposing sides of the barrier layer. The skin layer 30 can contain polypropylene homo-polymer, polypropylene random copolymer, polypropylene based TPO, polyamide (nylon), styrene-ethylene-butylene-styrene block copolymer, copolyester ether block copolymer, or a combination thereof. The peel seal layer 40 can contain a blend of a polypropylene random copolymer having a melting temperature greater than 140° C., a styrene-ethylene-butylene-styrene block copolymer, and an LLDPE having a melting temperature greater than 115° C.

As shown in FIG. 3, the film can further include a core layer 32 positioned between the skin layer 30 and the barrier layer 36. The core layer 32 can contain propylene-ethylene random copolymer, syndiotactic propylene-ethylene copolymer, polypropylene elastomer, polypropylene homopolymer, propylene based elastomer, ethylene based elastomer, styrene-ethylene-butylene-styrene block copolymer, ethylene-propylene rubber modified polypropylene and combinations thereof. Suitable propylene-ethylene copolymers include those sold by Exxon under the VISTAMAXX tradename, by Dow under the VERSIFY tradename, by Total under the ATOFINA tradename and by Basell under the PROFAX tradename. The film can further comprise one or more tie layers 34 and 38 that attach the skin layer 30, the peel seal layer 40, the barrier layer 36 and/or the core layer 34 to each other.

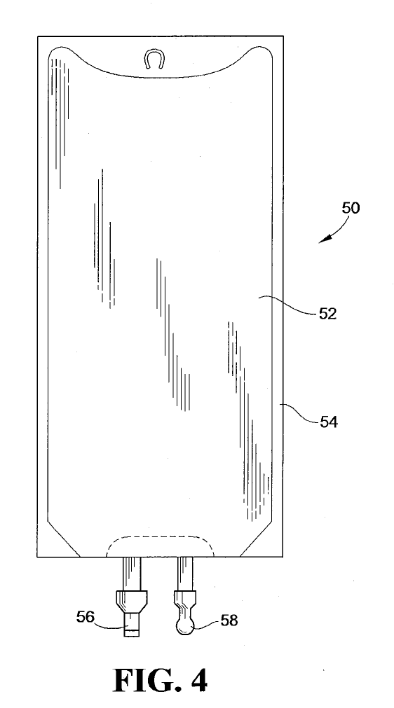

The films in embodiments of the present disclosure can be used to make any suitable containers, for example, used to hold a substance such as a pharmaceutical or a medical compounds or solution. In an embodiment shown in FIG. 4, the present disclosure provides a container 50 comprising a first sidewall 52 and a second sidewall (not shown) opposite the first sidewall sealed together along a peripheral seam 54 to define a fluid chamber. The container 50 can comprise one or more port tubes 56 and 58 that are used to fill and empty the contents of the container 50. Any one or more of the sidewalls can be fabricated from one of the monolayer or multiple layered films set forth above. It will also be appreciated that the container may be formed from an extruded tubular film sealed at its open ends. In this case, the peripheral seam 54 may consist of two seams on opposing ends of the tube. The container may be configured such that the seams are at the top and bottom of the container or along its vertical sides.

In an embodiment, the first sidewall and/or second sidewall is a film having at least one of 1) a peel seal layer comprising a blend of a random copolymer polypropylene having a melting temperature greater than 140° C., a styrene-ethylene-butylene-styrene block copolymer and an LLDPE having a melting temperature greater than 115° C.; 2) a peel seal layer comprising a blend of a random copolymer polypropylene having a melting temperature greater than 140° C. and an ethylene-propylene rubber modified polypropylene elastomer; and 3) a barrier layer comprising a caprolactam-free nylon compound.

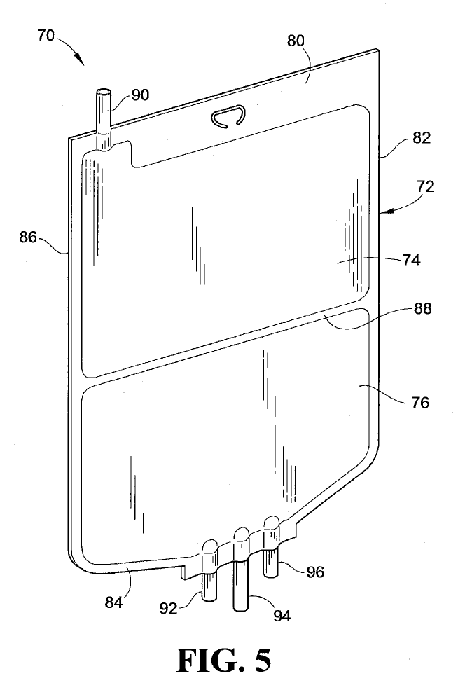

In an alternative embodiment shown in FIG. 5, the present disclosure provides a multiple chamber container 70 comprising a body 72 defined by a film. The multiple chamber container 70 comprises two chambers 74 and 76. It should be appreciated that in alternative embodiments more than two chambers can be provided in the container. The chambers 74 and 76 are designed for the separate storage of substances and/or solutions.

In the illustrated embodiment, any portion of the container 70 is made from a film having at least one of 1) a peel seal layer comprising a blend of a polypropylene random copolymer having a melting temperature greater than 140° C., a styrene-ethylene-butylene-styrene block copolymer and an LLDPE having a melting temperature greater than 115° C., 2) a peel seal layer comprising a blend of a polypropylene random copolymer having a melting temperature greater than 140° C. and an ethylene-propylene rubber modified polypropylene elastomer and 3) a barrier layer comprising a caprolactam-free nylon compound.

The container 70 may be made from two sheets of the film that are, for example, heat sealed along their edges (80, 82, 84, and 86) to form permanent seals. In the illustrated embodiment, two sheets of film are used. The sheets are sealed about the periphery of the container 70 at edges 80, 82, 84, and 86. Alternatively the container may be formed from an extruded tubular film sealed at its open ends. In this case, only two opposing edges of the container (for example edges 82 and 86) need to be sealed. A peelable seal 88 is provided between the sheets of film to form the chambers 74 and 76. Of course, if additional chambers are provided, additional peelable seals can be provided.

The container 70 and the peelable seal 88 can be constructed from films having a peel seal layer in accordance with embodiments of the present disclosure. The peel seal layer can allow both a peelable and permanent seal to be created. Thus, the permanent side seals 80, 82, 84, and 86 as well as the peelable seal 88 can be created from the same layer of film.

As illustrated in FIG. 5, the container 70 can further comprise one or more ports 90, 92, 94 and 96. The ports 90, 92, 94 and 96 provide communication with the interior of chambers 74 and 76, but could be located at any appropriate locations on container 70. These ports allow fluid to be added to or removed from the chambers 74 and 76. The ports 90, 92, 94 and 96 can also include a membrane (not shown) that is pierced by, for example, the cannula or spike of an administration set. It will be appreciated that one or more of the ports may be provided in the form of a molded structure with a surface specially adapted for sealing to the container, either between the sheets (in which case the port structure is sometimes referred to as a “gondola”) or directly to the wall. It will also be appreciated that the ports may include valves or similar closure structures rather than a simple membrane. Examples of such alternative port structures include the medication port depicted in U.S. Pat. No. 6,994,699 and the various access ports depicted in U.S. Patent Publication No. 2005/0083132, each of which is incorporated herein by reference, and

Depending on the methods employed to manufacture the containers, fill ports may not be necessary at all. For example, if the containers are to be manufactured from a continuous roll of plastic film, the film could be folded lengthwise, a first permanent seal created, the first compartment filled with solution, then a peelable seal created, a second compartment filled, a permanent seal created, and so on.

### EXAMPLES

By way of example and not limitation, the following examples are illustrative of various embodiments of the present disclosure.

### Example 1

Blends containing co-polypropylene, SEBS block copolymer and LLDPE were extruded as monolayer films. The seal performance of the post-autoclaved films was evaluated along with some other properties such as clarity (haze), tensile and autoclavability (via the observation on the surface appearance). Comparative testing of films that include a commercial product (CAWITON® PR4581A—comparative-1) and two films having a composition of 60%/25%/15% of co-polypropylene/SEBS/LLDPE (comparative-2 and comparative-3) was performed along with the formulated blends of the present disclosure. In addition, some of the peel seal layer blends of the present disclosure were coextruded with other layers to make multilayered films on which the peel seal performance was evaluated.

### I. Monolayer Films

TABLE 1
Comparison of different monolayer films (peak force, morphology)

| | Commercial | | | | | | | | | |
|---|---|---|---|---|---|---|---|---|---|---|
| | Product | | | | | | | | | |
| | (Cawiton) | | 31-3 | | | | | | | |
| | 31-1 & | | 34-3 | Formulations of the peel seal blends | | | | | | |
| | 34-1 & 36-1 | 31-2 & 34-2 | 36-2 | (PP + SEBS + LLDPE) | | | | | | |
| Formulation | (Comp -1) | (Comp. -2) | (Comp. -3) | 31-19 | 34-9 | 34-10 | 36-8 | 36-9 | 36-10 | 36-11 |
| Huntsman | | 60 | 60 | 70 | 75 | 70 | 75 | 75 | 70 | 70 |
| 43M5A | | | | | | | | | | |
| Kraton G | | 25 | 0 | 0 | 0 | 0 | 0 | 0 | 0 | 0 |
| 1652 | | | | | | | | | | |
| Kraton | | 0 | 25 | 25 | 17.5 | 22.5 | 17.5 | 17.5 | 22.5 | 22.5 |
| G1643 | | | | | | | | | | |
| Exxon | | 15 | 15 | 5 | 7.5 | 7.5 | 7.5 | 0 | 7.5 | 0 |
| LL3003 | | | | | | | | | | |
| Dowlex | | 0 | 0 | 0 | 0 | 0 | 0 | 7.5 | 0 | 7.5 |
| 2047G | | | | | | | | | | |
| TOTAL | | 100 | 100 | 100 | 100 | 100 | 100 | 100 | 100 | 100 |
| Peel Seal | | | | | | | | | | |
| (post | | | | | | | | | | |
| autoclaved) | | | | | | | | | | |
| Peak Force | | | | | | | | | | |
| (N/15 mm) | | | | | | | | | | |
| 120° C. | 1.0 | 0.7 | 2.0 | 1.0 | 0.7 | 0.7 | 0.7 | 0.7 | 1.7 | 1.2 |
| 130° C. | 7.1 | 1.3 | 3.2 | 2.5 | 4.7 | 2.2 | 0.8 | 1.1 | 3.8 | 4.8 |
| 140° C. | 36.3 | 11.1 | 27.7 | 19.2 | 26.5 | 25.4 | 25.8 | 30.7 | 24.3 | 31.1 |
| 150° C. | 44.8 | 39.0 | 43.2 | 48.6 | 50.5 | 50.7 | 60.3 | 61.1 | 53.8 | 56.1 |
| Peel Seal | | | | | | | | | | |
| Morphology | | | | | | | | | | |
| 120° C. | Adh. | Adh. | Adh. | Adh. | Adh. | Adh. | Adh. | Adh. | Adh. | Adh. |
| 130° C. | Adh. | Adh. | Adh. | Adh. | Adh. | Adh. | Adh. | Adh. | Adh. | Adh. |
| 140° C. | Adh. | Adh. | Adh. | Adh. | Adh. | Adh. | Adh. | Adh. | Adh. | Adh. |
| 150° C. | 4/5 RMS | Adh. | Adh. | Adh. | 2/5 Adh. | Adh. | 5/5 | 5/5 | 5/5 | 5/5 |
| | 1/5 RBS | | | | 3/5 RES | | Adh. | Adh. | Adh. | Adh |
| | | | | | | | 5/5 | 5/5 | 4/5 | 5/5 |
| | | | | | | | SMS | SMS | SMS | SMS |
| | | | | | | | 1/5 RES | 4/5 | 1/5 | |
| | | | | | | | | RES | SES | |
| | | | | | | | | | 3/5 | |
| | | | | | | | | | RES | |

TABLE 2
Comparison of different monolayer films (haze, tensile, surface appearance)

| | Commercial | | | | | | | | | |
|---|---|---|---|---|---|---|---|---|---|---|
| | Product | | | | | | | | | |
| | (Cawiton) | | 31-3 & | | | | | | | |
| | 31-1 & 34-1 & | 31-2 & | 34-3 & | | | | | | | |
| | 36-1 | 34-2 | 36-2 | 31-19 | 34-9 | 34-10 | 36-8 | 36-9 | 36-10 | 36-11 |
| Haze (post | | | | | | | | | | |
| autoclaved) | | | | | | | | | | |
| Haze (dry), % | 65.4 | 40.3 | 23.4 | — | 18.6 | 17.3 | 17.2 | 16.9 | 15.7 | 13.9 |
| Haze (wet on | 51.9 | 25.2 | 17.8 | — | 15.4 | 11.2 | 11.1 | 12.8 | 9.6 | 8.7 |
| seal-side), % | | | | | | | | | | |
| Haze (wet on | 18.8 | 10.0 | 10.3 | — | 6.3 | 6.2 | 5.6 | 3.9 | 4.4 | 3.1 |
| both sides), % | | | | | | | | | | |
| Tensile (post | | | | | | | | | | |
| autoclaved) | | | | | | | | | | |
| Young's | 45.7 | 55.5 | 42.4 | — | 57.4 | 56.8 | 63.6 | 61.9 | 50.7 | 47.0 |
| Modulus | | | | | | | | | | |
| (kpsi) | | | | | | | | | | |
| Yield Elong | 28.2 | 28.7 | 44.8 | — | 31.4 | 35.3 | 31.1 | 33.6 | 41.1 | 39.3 |
| (Z-Zlp) (%) | | | | | | | | | | |
| Yield | 1882 | 2335 | 1993 | — | 2483 | 2447 | 2508 | 2451 | 2257 | 2105 |
| Strength (Z- | | | | | | | | | | |
| Slp) (psi) | | | | | | | | | | |
| Tensile | 4342 | 5169 | 3284 | — | 4230 | 4081 | 4143 | 3981 | 4007 | 3772 |
| Strength (psi) | | | | | | | | | | |
| Break Ext (in) | 18 | 15 | 15 | — | 17 | 17 | 18 | 17 | 19 | 18 |
| Tensile Elong | 634 | 521 | 565 | — | 649 | 626 | 687 | 652 | 655 | 695 |
| (%) | | | | | | | | | | |
| Surface | OK | OK | OK | OK | OK | OK | OK | OK | OK | OK |
| Appearance | | | | | | | | | | |
| after | | | | | | | | | | |
| Autoclaving | | | | | | | | | | |

NOTE:
Adh.: Adhesive peel off
RBS, RMS, RES: Film ripped broken in the beginning, middle, end of the seal, respectively.
SMS, SES: Film stretched in the middle, end of the seal, respectively.

As shown in Tables 1 and 2, the peel seal blend formulations 31-19, 34-9, 34-10, 36, 8, 36-9, 36-10 and 36-11 were shown to have:
1. Wide range for the peel seal force: ˜3 to 30 N/15 mm
2. Low peel force at autoclaving temperature (121° C.): <3 N/15 mm
3. High permanent seal force than those of the three comparative formulas
4. Higher clarity than those of the three comparative formulas
5. Autoclavability

TABLE 3
Polymeric components of the peel seal blend

| | MFI, g/ | MFI, g/ | | |
|---|---|---|---|---|
| | 10 min | 10 min | | |
| | (190° C./ | (230° C./ | | |
| | 2.16 kg) | 2.16 kg) | Density | Chemistry |
| HUNTSMAN ® | — | 4.5 | 0.9 | random co-PP |
| 43M5A | | | | |
| KRATON ® | — | 5 | | SEBS |
| G1652 | | | | |
| KRATON ® | — | 18 | | SEBS with mid- |
| G1643 | | | | soft block having |
| | | | | high 1-2 addition |
| EXXON ® | 3.2 | — | 0.918 | ethylene-hexene- |
| LL3003 | | | | 1 copolymer |
| DOWLEX ® | 2.3 | — | 0.917 | ethylene-octene- |
| 2047 | | | | 1 copolymer |

II. Multilayer Films with No Gas Barrier Layer

Formula 36-9 used as the peel seal layer in a coextruded film, VistaPeel-2 (see Table 4). In contrast, the peel seal layers for Zcore-1 and Vista-1 are Comparative-2 and Comparative-3, respectively.

TABLE 4
Comparison of multilayer films with no gas barrier layer

| Sample | Skin Layer | | Core Layer | | Seal Layer | |
|---|---|---|---|---|---|---|
| Z CORE-1 | 0.6 | mil | 4.0 | mil | 3.4 | mil |
| | 90% | Total 6573XHC | ZELAS MC717 | | 60% | Huntsman 43M5A |
| | 10% | Kraton G1643 | | | 25% | Kraton G1652 |
| | | | | | 15% | EXXON LL3003 |
| VISTA-1 | 0.6 | mil | 6 | mil | 1.4 | mil |
| | 90% | Huntsman 43M5A | 83% | Vistamaxx 1100 | 60% | Huntsman 43M5A |
| | 10% | Kraton G1643 | 17% | Huntsman 43M5A | 25% | Kraton G1643 |
| | | | 15% | EXXON LL3003 | | |
| VistaPeel-2 | 0.6 | mil | 6 | mil | 1.4 | mil |
| | 90% | Huntsman 43M5A | 83% | Vistamaxx 1100 | 70% | Huntsman 43M5A |
| | 10% | Kraton G1643 | 17% | Huntsman 43M5A | 22.5% | Kraton G1643 |
| | | | 7.5% | Dowlex 2047G | | |
| FFS-14 | 1.5 | mil | 6 | mil | 2.7 | mil |
| | 50% | Zelas MC 717 | 77% | Vistamaxx 6102 | 70% | Huntsman 43M5A |
| | 45% | Huntsman P4G3Z-050F | 19% | Huntsman X01462 | 7.5% | Dowlex 2247G |
| | 5% | Profax PF611 | 4% | Kraton G1643 | 22.5% | Kraton G1643 |
| FFS-15 | 1.5 | mil | 5.5 | mil | 2.9 | mil |
| | 50% | Zelas MC 717 | 62.5% | Vistamaxx 6102 | 70% | Huntsman 43M5A |
| | 45% | Huntsman P4G3Z-050F | 25% | Zelas MC 717 | 7.5% | Dowlex 2247G |
| | 5% | Profax PF611 | 12.5% | Huntsman X01462 | 22.5% | Kraton G1643 |

| | | |
|---|---|---|
| Seal layer | ZCORE-1 = | Comparative-2 |
| of | VISTA-1 = | Comparative-3 |
| | VistaPeel-1 = | 36-9 |

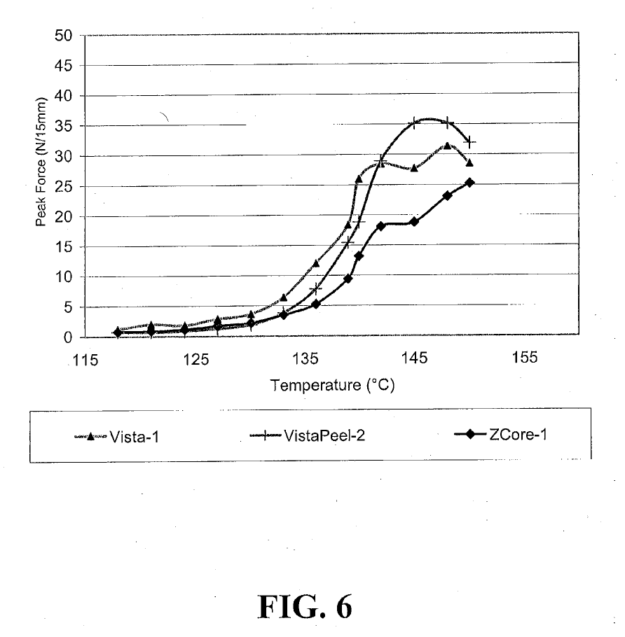

The peel seal performance for the multiple layer films is summarized in Table 5 and FIG. 6.

TABLE 5
Peel Force of the multilayer films of Table 4

| Peak Peel Force | | | | | |
|---|---|---|---|---|---|
| (N/15 mm) | Zcore-1 | Vista-1 | Vista Peel-2 | FFS-14 | FFS-15 |
| T (° C.) | | | | | |
| 118 | 0.8 | 1.2 | 0.7 | 1.1 | 0.6 |
| 121 | 0.9 | 2.0 | 0.7 | 1.0 | 0.6 |
| 124 | 1.1 | 1.8 | 0.9 | 0.9 | 0.6 |
| 127 | 1.7 | 2.9 | 1.2 | 1.2 | 0.6 |
| 130 | 2.2 | 3.7 | 1.9 | 1.2 | 0.7 |
| 133 | 3.5 | 6.4 | 3.8 | 1.7 | 0.9 |
| 136 | 5.2 | 12.0 | 7.8 | 2.5 | 1.4 |
| 139 | 9.4 | 18.4 | 15.4 | 3.5 | 2.3 |
| 140 | 13.2 | 26.0 | 18.8 | 4.4 | 6.8 |
| 142 | 18.1 | 28.5 | 28.9 | 14.4 | 13.3 |
| 145 | 18.8 | 27.7 | 35.1 | 19.9 | 18.2 |
| 148 | 23.1 | 31.3 | 35.1 | 34.1 | 27.7 |
| 150 | 25.2 | 28.5 | 31.9 | 44.1 | 43.8 |
| Peel Seal | | | | | |
| Morphology | | | | | |
| 118 | Adh. | Adh. | Adh. | Adh. | Adh. |
| 121 | Adh. | Adh. | Adh. | Adh. | Adh. |
| 124 | Adh. | Adh. | Adh. | Adh. | Adh. |
| 127 | Adh. | Adh. | Adh. | Adh. | Adh. |
| 130 | Adh. | Adh. | Adh. | Adh. | Adh. |
| 133 | Adh. | Adh. | Adh. | Adh. | Adh. |
| 136 | Adh. | Adh. | Adh. | Adh. | Adh. |
| 139 | Adh. | Adh. | Adh. | Adh. | Adh. |
| 140 | Adh. | Adh. | Adh. | Adh. | Adh. |
| 142 | Adh. | Adh. | Adh., SBS | 1/5 Adhesive, | 5/5 Adhesive |
| | | | | SW; 4/5 | |
| | | | | Adhesive | |
| 145 | Adh. | 2/5 Adh. | Adh., SBS, | 5/5 Adhesive, | 5/5 Adhesive |
| | | 3/5 RES | RBS | SW | |
| 148 | Adh. | Adh. | Adh. SBS | 4/5 SMS, SW; | Adhesive, SW, |
| | | | | 1/5 Adhesive, | 4/5; SES, 1/5 |
| | | | | SW | |
| 150 | Adh. | RMS | Adh., SBS, | 4/5 SMS, SW; | 5/5 Adhesive |
| | | | RBS | 1/5 SMS, RES, | |
| | | | | SW | |

Failure Mode:
Adh.: Adhesive peel off
SW: Stress whitening
SBS, SMS, SES: Film stretched in the beginning, middle, end of the seal, respectively.
RBS, RMS, RES: Film ripped broken in the beginning, middle, end of the seal, respectively.

Table 5 and FIG. 6 show that VistaPeel-2 has a low peel seal force near the autoclaving temperature, possesses a wide peel seal range (up to about 30 Nt/15 mm) and has the highest permanent seal force.

III. Multilayer Films with Gas Barrier Layer

Formula 36-9 was applied as the peel seal layer in a coextruded film: NylonPeel-2 (see Table 6). In contrast, the peel seal layers for Symredad and NB-1 are Comparative-1 and Comparative-3, respectively.

TABLE 6
Multilayer films with a gas barrier layer

| | | | Barrier | Tie | |
|---|---|---|---|---|---|
| Sample | Skin Layer | Tie Layer | Layer | Layer | Seal Layer |
| Symredad | 1.9 mil | 0.2 mil | 1.1 mil | 0.2 mil | 3.7 mil |
| | Borealis RE216CF | ADMER | EMS Grilon | ADMER | Cawiton PR4581A |
| | | QF300E | FG40NL | QF300E | |
| NB-1 | 1.8 mil | 0.4 mil | 1.1 mil | 0.4 mil | 3.5 mil |
| | 90% Huntsman 43M5A | ADMER | EMS Grilon | ADMER | 60% Huntsman 43M5A |
| | 10% Kraton G1643 | QB510A | BM20SBG | QB510A | 25% Kraton G1643 |
| | | | | | 15% EXXON LL3003 |
| NP-2 | 1.8 mil | 0.4 mil | 1.1 mil | 0.4 mil | 3.5 mil |
| | 90% Huntsman 43M5A | ADMER | EMS Grilon | ADMER | 70% Huntsman 43M5A |
| | 10% Kraton G1643 | QB510A | BM20SBG | QB510A | 22.5% Kraton G1643 |
| | | | | | 7.5% Dowlex 2047G |

| | | |
|---|---|---|
| Seal layer of | Symredad = | Comparative-1 |
| | NB-1 = | Comparative-3 |
| | NP-2 = | 36-9 |

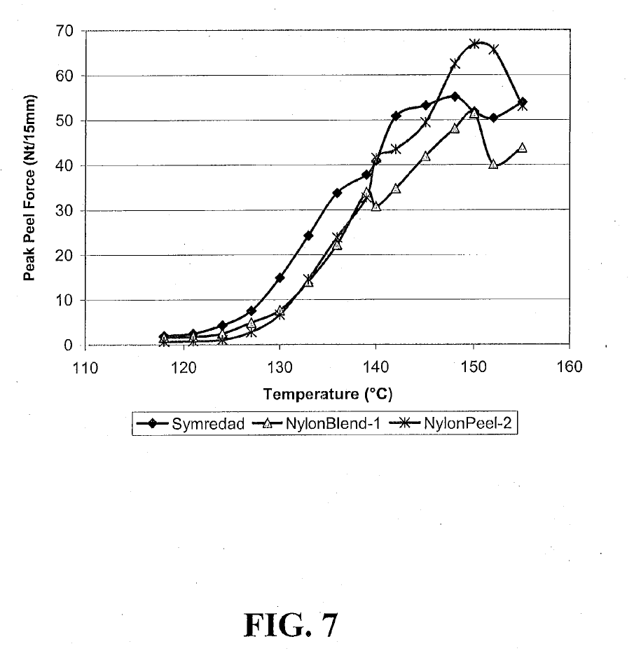

The peel seal performance for the multiple layer films is summarized in Table 7 and FIG. 7.

TABLE 7
Peel Force of the multilayer films of Table 6

Peak Peel Force (N/15 mm)
and Peel Seal Morphology

| T (° C.) | Symredad | NB-1 | NP-2 |
|---|---|---|---|
| 118 | 1.9 | 1.6 | 0.7 |
| | Adh. | Adh. | Adh. |
| 121 | 2.5 | 1.7 | 0.8 |
| | Adh. | Adh. | Adh. |
| 124 | 4.3 | 2.5 | 1.1 |
| | Adh. | Adh. | Adh. |
| 127 | 7.5 | 4.9 | 2.8 |
| | Adh. | Adh. | Adh. |
| 130 | 14.9 | 7.6 | 6.6 |
| | Adh. | Adh. | Adh. |
| 133 | 24.3 | 14.0 | 14.5 |
| | Adh. | Adh. | Adh. |
| 136 | 33.7 | 22.2 | 23.9 |
| | Adh. | Adh. | Adh. |
| | 1/5 Delam. | | |
| 139 | 37.8 | 33.9 | 32.7 |
| | Adh. | Adh. | Adh. |
| | 2/5 Delam. | | |
| 140 | 40.9 | 30.8 | 41.5 |
| | Adh. | Adh. | Adh. |
| | 1/5 Delam. | | |
| 142 | 50.8 | 34.7 | 43.4 |
| | Adh. | Adh. | Adh. |
| | 4/5 Delam. | | |
| 145 | 53.2 | 41.9 | 49.5 |
| | 5/5 Delam | Adh. | Adh. |
| 148 | 55.1 | 48.1 | 62.5 |
| | 5/5 Delam | Adh. | Adh. |
| | | | 2/5 SES |
| 150 | 51.8 | 51.4 | 66.8 |
| | 5/5 Delam | Adh. | Adh., SES |
| 152 | 50.4 | 40.1 | 65.6 |
| | 5/5 Delam | Adh. | 4/5 Adh. SES |
| | | | 1/5 SBS |
| 155 | 53.9 | 43.8 | 53.1 |
| | 5/5 Delam | Adh. | Adh., SES |

NOTE:
Adh.: Adhesive peel off
SBS, SES: Film stretched in the beginning and end of the seal, respectively.
Delam.: Delamination

Table 7 and FIG. 7 show that the NP-2 has a low peel seal force near the autoclaving temperature, possesses a wide peel seal range (up to about 50 N/15 mm) and has the highest permanent seal force (at the seal temperature between 147 to 155° C.).

### Example 2

Example 1 provides evidence that formulations comprising a blend of copolymer polypropylene/SEBS/LLDPE provide improved peel seal layers capable of being used in many applications. This study focused on improving the toughness of these films while maintaining peel seal characteristics by compounding elastomeric materials and/or lower melting polyolefins into appropriate layers of a multiple layer film. For example, the present formulations included materials that were compounded into the peel and/or skin layers of various multilayer coextruded films. In addition, PCCE (poly(cyclohexylene dimethylene cyclohexanedicarboxylate), glycol and acid comonomer) was also used as a tough skin layer in combination with newly compounded peel seal layers. The resulting structures were then tested for peel seal, haze, toughness using ASTM D3763, “High Speed Puncture Properties of Plastics Using Load and Displacement Sensors” and/or functional container drop testing.

In the current example, four different peel seal approaches were attempted and compared to the Cawiton baseline. Typical formulations for these peel seal layer approaches are given below.
1. Sample 1: This peel seal formulation comprised a blend of approximately 60% PP, 25% SEBS and 15% PE. The PP used melts at ˜145° C.
2. Sample 2: This peel seal formulation comprised a blend of approximately 60% TOTAL® 8573 PP, 25% SEBS and 15% LLDPE. The TOTAL® 8573 PP is a softer, slightly lower melting material (135° C.) that adds some toughness.
3. Sample 3: This peel seal formulation comprised a blend of approximately 60% Huntsman® 43M5A PP, 25% SEBS and 15% LLDPE. The Huntsman® 43M5A is a slightly higher melting PP (148° C.) to shift the peel seal curve to higher temperatures for Japanese peel requirements.
4. Sample 4: This peel seal formulation comprised a blend of approximately 60% HUNTSMAN® 43M5A PP, 25% KRATON® G1643 SEBS, which is more highly branched and more compatible with PP, and 15% LLDPE.
5. Sample 5: This peel seal formulation comprised a blend of approximately 70% Zelas 7023 (a polypropylene-based thermoplastic elastomer) and 30% Huntsman 43M5A. ZELAS® 7023 melts at ˜161° C., which allows the Japanese peel requirement to be easily met.

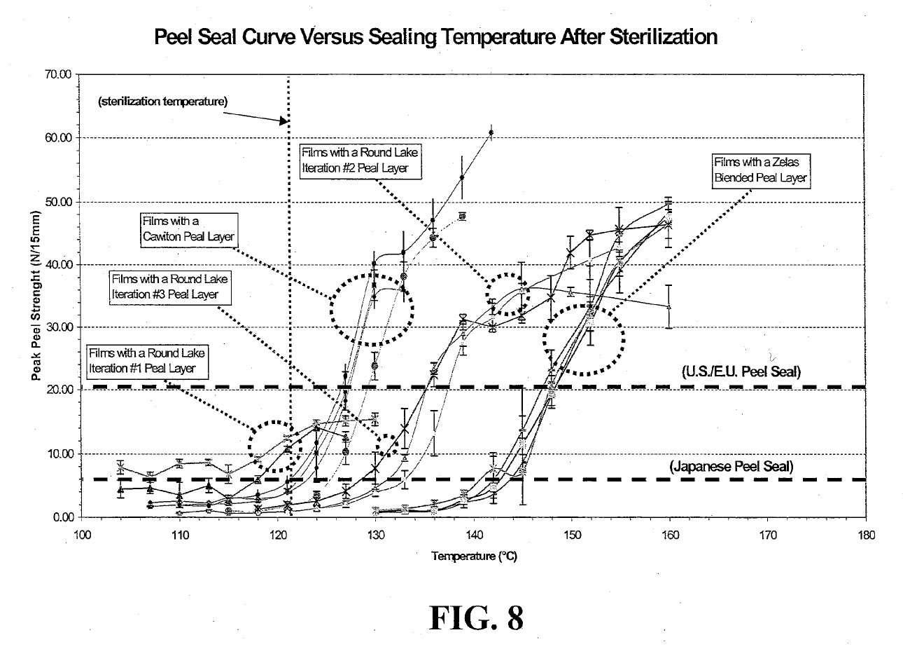

These peel seal layers were extruded in several multilayer structures with almost identical peel seal curve results. Typical peel seal curves for the different sample formulations are given in FIG. 8. FIG. 8 shows that Sample 3, Sample 4 and Sample 5 all provide peel seal characteristics that meet desired properties. These formulations can be used to generate a peel seal between about 4N/15 mm to about 30N/15 mm at temperatures greater than 121° C.

Alternative embodiments of three different iterations of films containing a nylon barrier layer and Sample 3, Sample 4 and Sample 5 peel seal layers were manufactured and tested. The first film iteration included structures with a Sample 3 peel layer and/or a PCCE skin layer and are shown in alternative embodiments of a five layer structures as illustrated in FIG. 2 having the following order: skin layer 20/tie layer 22/barrier layer 24/tie layer 26/seal layer 28. The details of the film layers are described in Table 8. The units at the end of each layer denote the thickness of that layer.

TABLE 8
First Iteration Film Formulations

| | TP-1 | TP-4 | PCCE-5 | PCCE-6 |
|---|---|---|---|---|
| Skin layer: | Borealis Re216CF PP, | | Eastman Ecdel 9966 PCCE, | |
| | 1.8 mil | | 1.8 mil | |
| Tie layer: | Dupont Bynel 4104, | Admer QF300E, | Dupont Bynel 4104 | |
| | 0.4 mil | 0.4 mil | 0.4 mil | |
| Barrier | EMS Grilon | EMS Grilon | EMS Grilon | EMS Grilon FG40NL, |
| layer: | BM20SBG, | FG40 NL, | BM20SBG, | 1.1 mil |
| | 1.1 mil | 1.1 mil | 1.1 mil | |

| | | | |
|---|---|---|---|
| Tie layer: | Dupont Bynel 4104, | Admer QF300E, | Dupont Bynel 4104 |
| | 0.4 mil | 0.4 mil | 0.4 mil |

| | | | |
|---|---|---|---|
| Seal layer: | 60% Huntsman 43M5A | 3PP/PE/SEBS | 60% Huntsman 43M5A |
| | 15% Exxon LL3003 | Cawiton Med | 15% Exxon LL3003 |
| | 25% Kraton G1652, | PR4581, | 25% SEBS Kraton G1652, |
| | 3.5 mil | 3.5 mil | 3.5 mil |

Alternative embodiments of the second film iteration included structures with a Sample 3 or Sample 4 peel layer and, in two of the structures, a PP/SEBS skin layer. The second iteration film structures include maleic anhydride modified homopolymer (ADMER® QF300E and QB510A) and copolymer (ADMER® 551A) tie layers. All embodiments of these film structures were five layer structures as illustrated in FIG. 2 having the following order: skin layer 20/tie layer 22/barrier layer 24/tie layer 26/seal layer 28. The details of the film layers are described in Table 9.

TABLE 9
Second Iteration Film Formulations

| | | | | |
|---|---|---|---|---|
| N-1 | N-2 | N-3 | N-4 | N-5 |

| | | | |
|---|---|---|---|
| Skin Layer: | Huntsman | 90% Huntsman 43M5A | Huntsman 43M5A, |
| | 43M5A, | 10% Kraton G1643 | 1.8 mil |
| | 1.8 mil | 1.8 mil | |

| Tie layer: | Admer QF300E, 0.4 mil | Admer | Admer |
|---|---|---|---|
| | | QF551A, | QB510A, |
| | | 0.4 mil | 0.4 mil |
| Barrier | EMS Grilon FG40 NL, 1.1 mil | | |
| layer: | | | |
| Tie layer: | Admer QF300E, 0.4 mil | Admer | Admer |
| | | QF551A, 0.4 mil | QB510A, 0.4 mil |

| | | | |
|---|---|---|---|
| Seal layer: | 60% PP Huntsman 43M5A | 60% PP | 60% PP Huntsman 43M5A |
| | 15% LLDPE Exxon LL3003 | Huntsman | 15% LLDPE Exxon LL3003 |
| | 25% SEBS Kraton G1652, | 43M5A | 25% SEBS Kraton G1652, |
| | 3.5 mil | 15% LLDPE | 3.5 mil |
| | | Exxon LL3003 | |
| | | 25% SEBS | |
| | | Kraton G1643, | |
| | | 3.5 mil | |

Alternative embodiments of the third iteration film structures included Sample 3, Sample 4 or Sample 5 blended seal layers along with PCCE or PP/SEBS skin layers. Also, PT-4 includes a maleic anhydride modified homopolymer PP blended with SEBS to further toughen the structure. All the third iteration film structures were five layer structures as illustrated in FIG. 2 having the following order: skin layer 20/tie layer 22/barrier layer 24 tie layer 26/seal layer 28. The details of the film layers are described in Table 10.

TABLE 10
Third Iteration Film Formulations

| | PT-1 | PT-2 | PT-3 | PT-4 | FGN-2 |
|---|---|---|---|---|---|
| Skin | 100% PCCE Eastman Ecdel | | 90% PP Huntsman 43M5A | | 90% PP |
| Layer: | 9966, | | 10% SEBS Kraton G1643, | | Huntsman |
| | 1.8 mil | | 1.8 mil | | XO1466 |
| | | | | | 10% SEBS |
| | | | | | Kraton G1643, |
| | | | | | 1.8 mil |
| Tie layer: | Bynel 4109, | | Admer QB510A, | 90% Admer | Modic P604V |
| | 0.4 mil | | 0.4 mil | QB510A | 0.4 mil |
| | | | | 10% SEBS Kraton | |
| | | | | G1643, | |
| | | | | 0.4 mil | |
| Barrier | 100% PA EMS Grilon FG40 NL, 1.1 mil | | | | |
| layer: | | | | | |
| Tie layer: | Admer QB510A, | | | 90% Admer | Modic P604V |
| | 0.4 mil | | | QB510A | 0.4 mil |
| | | | | 10% SEBS Kraton | |
| | | | | G1643, | |
| | | | | 0.4 mil | |
| Seal layer: | 60% PP | 70% Zelas | 60% PP | 60% PP Huntsman | 70% PP |
| | Huntsman | 7023 | Huntsman | 43M5A | Huntsman |
| | 43M5A | 30% PP | 43M5A | 15% LLDPE | XO1466 |
| | 15% LLDPE | Huntsman | 15% LLDPE | Exxon LL3003 | 22.5% SEBS |
| | Exxon | 43M5A, | Exxon LL3003 | 25% SEBS Kraton | Kraton G1643 |
| | LL3003 | 3.5 mil | 25% SEBS | G1652, | 7.5% Dowlex |
| | 25% SEBS | | Kraton G1643, | 3.5 mil | 2247G |
| | Kraton | | 3.5 mil | | 3.5 mil |
| | G1652, | | | | |
| | 3.5 mil | | | | |

The results of impact testing and haze are shown in Table 11. The results showed the films containing the embodiments of Iteration #2 and #3 with the standard EMS FG40NL nylon (TP-4 and N-1 thru N-5) have improved impact toughness over a commercial Maestro film. The results also showed the film containing a PCCE skin layer and the standard EMS FG40NL nylon also have improved impact toughness over the Maestro film.

TABLE 11
Impact and Haze Properties of Barrier Films

| | Haze | | | | | |
|---|---|---|---|---|---|---|
| | | Haze | Impact | | | |
| | Haze | (wet on seal- | Maximum | Norm. energy at | Norm. energy at | |
| Film Name | (dry), % | side), % | load, kN | Max, J/mm | F, J/mm | Morphology |
| Maestro | 55.9 | 25.8 | 0.131 | 6.64 | 6.95 | Ductile |
| (lot: 060203T) | | | | | | |
| (7.1 mils) | | | | | | |
| PCCE-5 | 30.6 | 19.2 | 0.119 | NA | 5.82 | Ductile |
| PCCE-6 | 16.7 | 10.0 | 0.136 | 8.66 | 9.04 | Ductile |
| ToughPeel-1 | 20.0 | 13.7 | 0.131 | NA | 6.66 | Ductile |
| ToughPeel-4 | 21.5 | 19.4 | 0.149 | NA | 7.99 | Ductile |
| Nylon-1 | 21.8 | 14.9 | 1.620 | 8.00 | 8.30 | Ductile |
| Nylon-2 | 21.3 | 15.0 | 1.550 | 7.60 | 8.00 | Ductile |
| Nylon-3 | 19.6 | 16.9 | 1.590 | 7.90 | 8.30 | Ductile |
| Nylon-4 | 26.5 | 20.2 | 1.470 | 6.90 | 7.70 | Ductile |
| Nylon-5 | 24.4 | 18.4 | 1.520 | 7.80 | 8.20 | Ductile |
| PTie-1 | 14.2 | 7.0 | 1.500 | 7.30 | 7.70 | Ductile |
| PTie-2 | 12.2 | 6.0 | 1.370 | 7.40 | 7.80 | Ductile |
| PTie-3 | 18.3 | 13.6 | 1.430 | 7.30 | 7.50 | Ductile |
| PTie-4 | 21.2 | 16.7 | 1.360 | 6.70 | 7.10 | Ductile |

Based on the material compounds created and the film structures produced and results measured, novel peel seal compounds as well as multilayer films were developed. Examples of these peel seal compounds and film structures are given as follows:

In alternative embodiments, the peel seal layer film is capable of producing a seal by heated dies in multilayer extruded films that can be peeled apart without producing residual debris. By varying the temperature a peel force between 3N/15 mm and 30N/15 mm should be able to be created on the same peel layer compound in a variety of film structures and thicknesses. Peel seals should be created at temperatures greater than 122° C. The seal layer should be capable of sterilization at 121° C. without adversely affecting the peel force. One example of such a material is 60% PP random copolymer having a melting temperature greater than 145° C., 25% SEBS and 15% LLDPE having a melting temperature greater than 120° C. A second example is a blend of 60%-80% PP based TPO such as Zelas 7023 with 20%-40% random copolymer PP having a melting temperature greater than 130° C. A third example is a blend of 70% PP random copolymer having a melting temperature greater than 145° C., 22.5% SEBS and 7.5% LLDPE having a melting temperature greater than 120° C.

The previously described embodiments are directed to a tough and clear multilayer film containing a peel seal layer. The dart impact resistance of the film was shown to give a good correlation to container damage resistance in products. In one embodiment, the desired dart impact resistance is greater than 7 J/mm for the multilayer film. In addition it is desirable to maintain haze less than 20% for the film wetted on one surface. Finally, it is desired to have a CO₂ permeability for such a film to be less than 200 cm³/m² day atm. Examples of such films are TP-4, N-1, N-3, N-4, N-5, PT-1 PT-3, and FGN-2.

### Example 3

A caprolactam-free nylon-6,6/6,10 copolymer (BM20SBG from EMS-Grivory) has been found to be a good candidate from an extrusion standpoint for multilayer barrier films. However, films based on this structure show significantly inferior drop resistance, dart impact properties, and gas (O₂ and CO₂) permeability than current films containing nylon-6 based barrier layers. Amorphous nylon inherently has significantly improved gas barrier properties. (A minimum blend level to provide adequate gas barrier resistance can be calculated for a given grade using permeability data and a rule of mixtures.) Accordingly, the approach of this study was to blend amorphous nylon at appropriate levels with nylon-6,6/6,10 copolymer to improve the impact resistance and gas barrier resistance while maintaining acceptable clarity, as well as UV absorbance that is acceptable with global medical regulatory requirements.

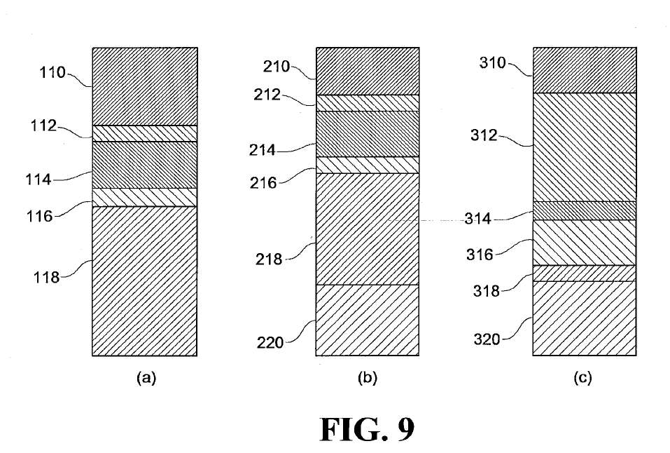

Amorphous nylon was blended with the nylon-6,6/6,10 copolymer and extruded as monolayers to find the best balance of impact resistance, clarity and permeability. Promising blends were identified and incorporated into one or more of the following film structures shown in FIGS. 9(a)-9(c). FIG. 9(a) is directed to a multiple layer film structure having the following order: skin layer 110/tie layer 120/barrier layer 130/tie layer 140/seal layer 150. FIG. 9(b) is directed to a multiple layer film structure having the following order: skin layer 210/tie layer 220/barrier layer 230/tie layer 240/core layer 250/seal layer 260. FIG. 9(c) is directed to a multiple layer film structure having the following order: skin layer 310/core layer 320/tie layer 330/barrier layer 340/tie layer 350/seal layer 360. Permeability, physical properties, and/or drop resistance of the multilayer films were then measured.

### Current Results

Small-scale process blending trials were conducted of available grades of amorphous nylon, which included EMS GRIVORY® G21 (nylon 6I/6T), EMS GRIVORY® HB5299 (nylon MXD6/MXDI copolymer), EMS GRIVORY®B HB7103 (same), and Dupont SELAR PA (nylon 6I/6T). EMS GRIVORY® HB7103 amorphous nylon was found to have the best combination of clarity, permeability resistance and mechanical properties. Monolayer films were then made using a 50%:50% and 85%:15% blend of EMS GRILON® BM20SBG nylon-6,6/6,10 copolymer and EMS GRIVORY® HB7103 amorphous nylon. The haze, dart impact and predicted permeability of these monolayers were then compared to the baseline EMS FG40NL, which is based on nylon-6, and BM20SBG. The results of this comparison are given in Table 12 below. In Table 12, permeability was calculated at different relative humidity conditions based on a rule of mixtures using available supplier data or measured Baxter data as available. Past industrial experience has also shown that the permeability for CO₂ is approximately 4 times higher than O₂.

The results in Table 12 show that the best blend was the 85%:15% blend of BM20SBG and HB7103. This blend had approximately twice the dart impact resistance of pure BM20SBG and predicted permeability almost equivalent to the FG40NL nylon currently used in Baxter's Maestro film. The haze of the 85%:15% blend was higher than either pure compound but still acceptable for use in multilayer films. When the blend ratio was changed to 50%:50%, there was no improvement in dart impact properties and an unacceptable increase in haze, as the monolayer film then appears cloudy. Optimization of the blend ratio was possible but given the monolayer properties the 85%:15% blend is satisfactory for current applications. Further testing revealed that adjusting the ratio to 87.5% BM20SBG/12.5% HB7103 provided somewhat better performance.

TABLE 12
Properties of different nylons and nylon blends

| | #1 | #2 | #5 | #8 | #10 |
|---|---|---|---|---|---|
| Formula (wt %) | | | | | |
| Grilon F40NL | 100 | 0 | 0 | 0 | 0 |
| Grilon BM 20 SBG | 0 | 100 | 0 | 50 | 85 |
| Grivory HB7103 | 0 | 0 | 100 | 50 | 15 |
| TOTAL | 100 | 100 | 100 | 100 | 100 |
| Impact, RT | | | | | |
| Norm. Energy at Max | 19.5 | 2.6 | *a | 3.6 | 5.0 |
| Load, J/mm | | | | | |
| Norm. Energy to | 26.1 | 3.1 | *a | 4.1 | 6.0 |
| failure, J/mm | | | | | |
| Morphology | 5/5 ductile | 1/5 ductile, | *a | 5/5 brittle | 1/2 ductile, 1/2 |
| | | 4/5 brittle | | | brittle |
| Appearance after | Clear | Clear | | Cloudy | Clear |
| autoclaving | | | | | |
| Wavy index, 1 = no | 3 | 3 | *a | 3 | 3 |
| wavy, 3 = control (40-10), | | | | | |
| 5 = worst | | | | | |
| O₂ Permeability | | | | | |
| (cm3/m2 day | | | | | |
| 25 um bar) | | | | | |
| Measured @ 0% r.h. | 56^{b} | 110^{b} | 20^{b} | | |
| Predicted @ 0% r.h. | | | | 34 | 66 |
| Measured @ 40% r.h. | 24^{b} | NA | 16^{b} | | |
| Predicted @ 40% r.h. | | | | TBD | TBD |
| Measured @ 85% r.h. | 56^{b} | 150^{b} | 20^{b} | | |
| Predicted @ 85% r.h. | | | | 35 | 76 |
| CO₂ Permeability | | | | | |
| (cm3/m2 day | | | | | |
| 25 um bar) | | | | | |
| Measured @ 40% r.h. | TBD | TBD | NA | NA | NA |
| Haze | | | | | |
| Haze (dry), % | 7.5 | 6.3 | 1.6 | 19.4 | 11.8 |
| Haze (wet on seal-side), % | 5.4 | 5.4 | 1.2 | 19.0 | 11.8 |
| Haze (wet on both | 4.3 | 3.7 | 0.9 | 18.3 | 11.2 |
| sides), % | | | | | |

*a = not autoclavable as a monolayer
^{b}= data from EMS-Grivory data sheets and public presentation

A study was completed comparing the 85%:15% blended nylon to pure BM20SBG or FG40NL in a five-layer coextruded film structure. The structures of the studied films are five-layer structures as illustrated in FIG. 2 having the following order: skin layer 20/tie layer 22/barrier layer 24/tie layer 26/seal layer 28. The structures include PT-3 as described in Example 2 along with the following structures shown in Table 13.

TABLE 13
Film formulations

| | NB-1 | CF-1 | CF-2 |
|---|---|---|---|
| Skin Layer: | 90% PP Huntsman 43M5A | | |
| | 10% SEBS Kraton G1643, | | |
| | 1.8 mil | | |
| Tie layer: | Admer QB510A, | | Modic P604V, |
| | 0.4 mil | | 0.4 mil |
| Barrier | 100% PA EMS Grilon | 85% PA Grilon BM20SBG | |
| layer: | BM20SBG, 1.1 mil | 15% PA Grivory HB7103, | |
| | | 1.1 mil | |
| Tie layer: | Admer QB510A, | Admer QB510A, | Modic P604V, |
| | 0.4 mil | 0.4 mil | 0.4 mil |

| | | |
|---|---|---|
| Seal layer: | 60% PP Huntsman | 70% PP Huntsman 43M5A |
| | 43M5A | 7.5% LLDPE Dowlex 2247G |
| | 15% LLDPE Exxon | 22.5% SEBS Kraton G1643, |
| | LL3003 | 3.5 mil |
| | 25% SEBS Kraton | |
| | G1643, 3.5 mil | |

Results of haze and impact testing are given in Table 14. The haze and impact of the CF-1 & CF-2 films, which contain the 85%:15% nylon blend, were better than NB-1, which contains pure BM20SBG in a similar structure. The impact resistance of CF-2 was better than CF-1 because it contains a polypropylene homopolymer-based tie layer rather than a copolymer-based tie layer, consistent with the trend that has been observed in previous work. The haze of CF-3 was also significantly better than the commercially available Maestro, which contains FG40NL, and the impact is almost equivalent. Both the haze and impact of PT-3, which contains FG40NL in a similar structure, were better than CF-3.

TABLE 14
Properties of Five-Layer Nylon Barrier Films with Different Nylon

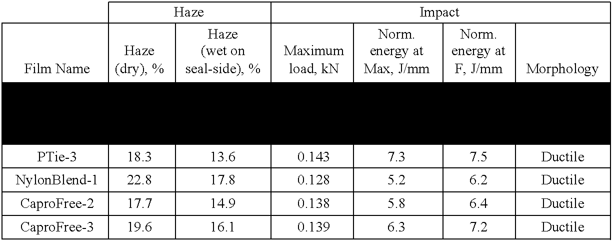

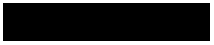

The six-layer structures shown in FIGS. 9(b) and 9(c) have shown to have better impact resistance than five-layer structures. Sample multiple-layer film structures that were extruded are given in FIG. 3. The multiple layer film structures have the following order: skin layer 30/core layer 32/tie layer 34/barrier layer 36/tie layer 38/seal layer 40. The details of the film layers are described in Table 15.

TABLE 15
Six-Layer Film formulations

| | CF-4 | CF-5 | CF-6 | CF-7 | CF-21 | ZN-1 | ZN-2 |
|---|---|---|---|---|---|---|---|
| Skin | 50% Zelas 717 | | | 50% Zelas | 90% | 50% Zelas | 50% Zelas |
| Layer: | 50% PP Huntsman P4G3Z-050F, | | | 717 | Huntsman | 717 | 717 |
| | 0.5 mil | | | 50% PP | XO1466 | 50% PP | 50% PP |
| | | | | Huntsman | 10% Kraton | Huntsman | Huntsman |
| | | | | P4G3Z, | G1643, | P4G3Z- | P4G3Z, |
| | | | | 0.5 mil | 1.0 mil | 050F, | 0.5 mil |
| | | | | | | 0.5 mil | |
| Core | 77% | 70% Infuse | 77% Vistamaxx 6102 | | 77% | 100% Zelas 717, | |
| layer: | Vistamaxx | 9007 | 19% PP Huntsman | | Vistamaxx | 4.0 mil | |
| | 6102 | 30% PP | XO1462 | | 6102 | | |
| | 19% PP | Huntsman | 4% Kraton G1643, | | 19% PP | | |
| | Huntsman | 43M5A, | 4.0 mil | | Huntsman | | |
| | XO1462 | 4 mil | | | XO1466 | | |
| | 4% Kraton | | | | 4% Kraton | | |
| | G1643, | | | | G1643, | | |
| | 4.0 mil | | | | 4 mil | | |
| Tie | Modic P604V, | | | Admer | Modic P604V, | | Admer |
| layer: | 0.4 mil | | | QB510A, | 0.4 mil | | QB510A, |
| | | | | 0.2 mil | | | 0.2 mil |
| Barrier | 85% PA Grilon BM20SBG | | | | 87.5% PA | 85% PA Grilon | |
| layer: | 15% PA Grivory HB7103, | | | | Grilon | BM20SBG | |
| | 1.1 mil | | | | BM20SBG | 15% PA Grivory | |
| | | | | | 12.5% PA | HB7103, 1.1 mil | |
| | | | | | Grivory | | |
| | | | | | HB7103, | | |
| | | | | | 1.1 mil | | |
| Tie | Modic P604V, | | | Admer | Modic P604V, | | Admer |
| layer: | 0.4 mil | | | QB510A, | 0.4 mil | | QB510A, |
| | | | | 0.2 mil | | | 0.2 mil |
| Seal | 70% PP | 70% PP | 75% PP | 70% PP | 70% PP | 100% | 100% |
| layer: | Huntsman | Huntsman | Huntsman | Huntsman | Huntsman | Zelas | Zelas |
| | XO1462 | XO1462 | XO1462 | XO1462 | XO1466 | 7023, | 7023 |
| | 7.5% | 7.5% | 25% Versify | 7.5% | 7.5% | 1.4 mil | 1.8 mil |
| | LLDPE | LLDPE | DE3300, | LLDPE | LLDPE | | |
| | Dowlex | Dowlex | 1.4 mil | Dowlex | Dowlex | | |
| | 2247G | 2247G | | 2247G | 2247G | | |
| | 22.5% | 22.5% | | 22.5% | 22.5% | | |
| | SEBS | SEBS | | SEBS | SEBS | | |
| | Kraton | Kraton | | Kraton | Kraton | | |
| | G1643, | G1643, | | G1643, | G1643, | | |
| | 1.4 mil | 1.4 mil | | 1.8 mil | 2.0 mil | | |

Based on the material compounds created, film structures produced, and results measured, novel five and six layer or more nylon barrier film structures can be made incorporating a caprolactam-free nylon blend that meet desired container properties. In an embodiment, the desired dart impact resistance is greater than 4.5 J/mm for the nylon barrier material to be used in a multilayer film. At the same time the nylon barrier layer should have good heat resistance to heat seal temperatures greater than 130° C. and haze less than 15% when wetted on both sides. Finally, the O₂ permeability should be less than 80 cm³/m² day 25 um bar at approximately 85% r.h. (relative humidity).

Descriptions of multiple layer films in alternative embodiments incorporating a caprolactam-free nylon barrier layer and their desired properties are as follows:

In an embodiment, the multiple layer film is a five-layer film as shown in FIG. 9(a) having a caprolactam-free nylon barrier layer. The film can have a CO₂ permeability less than 200 cm³/m² day atm. The film can also comprise peel seals that can be created between 4N/15 mm and 30N/15 mm by being heated at temperatures greater than 122° C. Dart impact resistance of the film has shown to give a good correlation to container damage resistance in products. The desired dart impact resistance can be greater than 6 J/mm for the multilayer film. In an alternative embodiment, it is desirable to maintain haze less than 20% for the film wetted on one surface. One example of such a film is CF-3.

In another embodiment, the multiple layer film is a six-layer film as shown in FIGS. 9(b)-9(c) having a caprolactam-free nylon barrier layer and tough core. The film can have a CO₂ permeability less than 200 cm³/m² day atm. The film can also comprise peel seals that can be created between 4N/15 mm and 30N/15 mm by being heated at temperatures greater than 122° C. Dart impact resistance of the film has shown to give a good correlation to container damage resistance in products. The desired dart impact resistance can be greater than 8 J/mm for the multilayer film. In an alternative embodiment, it is desirable to maintain haze less than 20% for the film wetted on one surface. Examples of such films are CF-4 through CF-7.

The multiple layer films can also comprise raw materials that do not contain substances (e.g. calcium or magnesium stearate, erucamide, other fatty acids, etc.) that can be leached from the film and/or precipitate to cause particulate matter in a solution having a between a pH ranging from 2 and 10.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A film comprising a barrier layer comprising a caprolactam-free nylon compound.

2. The film of claim 1, wherein the caprolactam-free nylon compound comprises a blend of about 75% to about 95% by weight of a polyamide 6,6/6,10 copolymer and about 5% to about 25% by weight of amorphous polyamide.

3. The film of claim 1, wherein the caprolactam-free nylon compound comprises a blend of about 87.5% by weight of a polyamide 6,6/6,10 copolymer and about 12.5% by weight of amorphous polyamide.

4. The film of claim 2 or claim 3, wherein the amorphous polyamide is selected from the group consisting of polyamide MXD6/MXDI copolymers, polyamide 6I/6T copolymers, and combinations thereof.

5. The film of claim 1 further comprising a skin layer and a peel seal layer, the skin layer and the peel seal layer attached to the barrier layer on opposing sides of the barrier layer.

6. The film of claim 5, wherein the skin layer comprises a component selected from the group consisting of polypropylene random copolymers, polypropylene homopolymers, nylon, styrene-ethylene-butylene-styrene block copolymer, copolyester ether block copolymers, and combinations thereof.

7. The film of claim 5, wherein the peel seal layer comprises a blend of a random copolymer polypropylene having a melting temperature greater than 140° C., a styrene-ethylene-butylene-styrene block copolymer and a linear low-density polyethylene having a melting temperature greater than 115° C.

8. The film of claim 5 further comprising at least one tie layer that attaches at least one of the skin layer and the peel seal layer to the barrier layer.

9. The film of claim 8, wherein the tie layer comprises a component selected from the group consisting of maleated linear low-density polyethylene, maleated polypropylene homopolymers, maleated polypropylene copolymers and combinations thereof.

10. The film of claim 5 further comprising a core layer positioned between the skin layer and the barrier layer.

11. The film of claim 5 further comprising a core layer positioned between the barrier layer and the peel seal layer.

12. The film of claim 10 or claim 11, wherein the core layer comprises a component selected from the group consisting of polypropylene homopolymers, propylene-ethylene random copolymers, syndiotactic propylene-ethylene copolymers, polypropylene elastomers, propylene based elastomers, ethylene based elastomers, styrene-ethylene-butylene-styrene block copolymers, ethylene-propylene rubber modified polypropylenes and combinations thereof.

13. The film of claim 10 or claim 11, wherein the film comprises a CO₂ permeability of less than 200 cm³/m² day atm.

14. The film of claim 10 or claim 11, wherein the film has a dart impact resistance greater than 6 J/mm when measured according to ASTM D3763.

15. The film of claim 10 or claim 11, wherein the film has less than 20% haze when wetted on one surface.

16. A container comprising:
a body defined by a film comprising a barrier layer, said barrier layer comprising a caprolactam-free nylon compound.

17. A multiple chamber container comprising:
a body defined by a film, the body including at least two chambers separated by a peelable seal, the film comprising a barrier layer comprising a caprolactam-free nylon compound.
